(19) United States
(12) Patent Application Publication
(10) Pub. No.: US 2026/0251555 A1
(43) Pub. Date: Aug. 27, 2026

(54) METHOD AND SYSTEM FOR ANALYSING ORE

(71) Applicants: ANGLO AMERICAN TECHNICAL & SUSTAINABILITY SERVICES LTD, London (GB); ANGLO CORPORATE SERVICES SOUTH AFRICA (PTY) LTD, Rosebank (ZA)

(72) Inventors: Daniel Clark FINFER, London (GB); Colin Murray ANDREW, Rosebank (ZA)

(21) Appl. No.: 18/875,021

(22) PCT Filed: Jun. 15, 2023
(86) PCT No.: PCT/IB2023/056213

§ 371 Date: Dec. 13, 2024

(30) Foreign Application Priority Data
Jun. 20, 2022 (GB) 2209021.1

(51) Int. Cl. G01N 21/27; E02F 9/26; G01N 21/25; G01N 33/24

(52) U.S. Cl. CPC G01N 21/27; E02F 9/261; G01N 21/255; G01N 33/24

(57) ABSTRACT

A method of analysing ore during excavation, wherein the method includes, during excavation, scanning/sensing ore by using an active hyperspectral sensing (AHS) sensor. The scanning/sensing step includes (i) controlling a power spectrum of a light source, and (ii) comparing, by using the AHS sensor, an emitted light spectrum of light emitted from the controlled light source with a light spectrum of reflected/backscattered light which is reflected/backscattered off the ore. The AHS sensor may be directed towards a muck/veneer/stockpile in order to scan ore forming part of, or being located on, the muck/veneer/stockpile.

### REFERENCES

AU 2020273300 relates to a mining shovel with compositional sensors which are installed inside a mining shovel bucket. These sensors may be photometric, radiometric or electromagnetic sensors.

EP 2 844 987 relates to a system and method of sorting mineral streams, for example laterite mineral ores, into appropriately classified valuable and waste streams for maximum recovery of value from the mineral stream. The method includes receiving response data indicating reflected, absorbed or backscattered energy from a mineral sample exposed to a sensor, where the mineral sample is irradiated with electromagnetic energy. The sensor may be an X-ray fluorescence sensor.

AU 2012277493 relates to a method of analyzing minerals received within a mining shovel bucket. The method includes collecting data associated with ore received in the bucket, where the bucket includes at least one active sensor which is installed in the bucket itself.

WO2021/108838 relates to a method for detecting changes in the ore grade of a rock face in near real time. The system uses a passive hyperspectral sensor which uses environmental light or an artificial light during operation. WO 2009/109006 relates to a scanning module which comprises hyperspectral cameras to scan an ore body to make ore grade assessments. The system described in the document utilises passive hyperspectral sensing.

WO 2016/112430 discloses the use of a passive hyperspectral imager that can be used to scan mineral ores.

WO 2014/134655 discloses a survey vehicle with a passive hyperspectral camera, which operates in a mining environment.

JP 2022043894 A discloses a method for discriminating ore according to the arsenic content using a passive hyperspectral camera.

### BACKGROUND OF THE INVENTION

This invention relates to a method and system for analysing ore.

Bulk ore sorting (BOS) involves the use of sensing technology to reject waste ore prior to minerals processing for the purpose of reducing the energy intensity of the mining process and optimising the cost expended per tonne of metal produced. In conventional BOS, sensors are installed after the primary crusher to detect the grade of ore on the conveyor belt. A threshold can then be applied, making it possible to identify low grade ores and reject them to the waste pile or stockpile.

In other implementations, grade data is combined with other data, such as mineralogy or physical data, to determine the value of the ore, enabling Value-Based Ore Control (VBOC) which is more sophisticated than grade-based ore control. This mineralogical and physical data can be taken from the block model or, preferably, measured in real time in the post-crusher environment. In other implementations, sensor data from the belt can be used to stabilise the plant by determining the level of deleterious mineral present within a given sample. This plant stability method can be enhanced through the use of stockpiles. An example of stockpile-driven stability might be encountered in copper mining where Arsenic is highly variable within the pit. A concentrate marketing philosophy might be developed to capitalise on stabilised Arsenic control, requiring the continual measurement and blending of feed to the plant for the purpose of targeting a specific do-not-exceed arsenic level within the concentrate.

BOS to date has relied primarily on the use of on-belt sensors. These sensors come in a variety of types, and are defined typically by the geometric portion of the ore contributing to the output (volume or surface) and the type of data provided (chemical, mineralogical or physical). Volume sensors, which make it possible to represent the ore on a volume basis, offer the best representativity but are usually expensive nuclear devices (e.g. PFTNA (Pulsed fast and thermal neutron activation) or PGNAA (Prompt gamma neutron activation analysis)) or specialist systems available from a limited pool of suppliers (e.g. NMR (Nuclear Magnetic Resonance) systems). Surface sensors of several types (e.g. XRF, LIBS, or hyperspectral) are widely available from a large pool of suppliers, but can only measure what they can “see”, leading to lower representativity in typical installations in comparison to volume sensors. In terms of output type, chemical data can be used to determine grade, while mineralogical systems provide a picture of the actual type of rock present. Physical measurements can present many types of data (hardness, texture, density) and are important for contextualising both chemical and mineralogical data.

Up to now, the market has only presented one credible solution for sensing during excavation. This solution is based on XRF (X-ray fluorescence) technology and makes it possible to measure elemental composition of ore within the bucket during mining. This technology however has four primary technical drawbacks: (1) XRF sensing is limited to the measurement of surface properties, (2) XRF output is limited to chemical composition, (3) the existing XRF solution requires the sensors to be mounted in/on the mining buckets (owning to proximity requirements) which make them vulnerable to damage as rocks are being loaded into the bucket during operation, and (4) the proximity limits for this technology mean that there is likely no pathway to enabling the technology to future applications for scanning of the veneer during mining.

The inventors wish to address at least some of the problems mentioned above.

### SUMMARY OF THE INVENTION

In accordance with a first aspect of the invention there is provided a method of analysing ore during excavation, wherein the method includes:
during excavation, scanning/sensing ore by using an active hyperspectral sensing (AHS) sensor.

The scanning/sensing step may include controlling an operation of a light source (hereinafter referred to as the “controlled light source”).

The scanning/sensing step may more specifically include:
a. controlling a power spectrum of the controlled light source; and
b. comparing, by using the AHS sensor, an emitted light spectrum of light emitted from the controlled light source with a light spectrum of reflected/backscattered light which is reflected/backscattered off the ore.

The comparison step may include comparing spectral features and/or brightness of the emitted light spectrum with spectral features and/or brightness of the reflected/backscattered light.

The method may include using the controlled light source to emit light towards the ore.

The controlled light source may form part of the AHS sensor.

The controlled light source may be a supercontinuum beam. The controlled light source may be temporally variant to improve signal fidelity. The emitted light or light signal may be encoded to enable the use of correlation methods when comparing an emitted signal or emitted light with a received signal or a received light.

The scanning/sensing step may include:
a. controlling an operation of the controlled light source, which includes controlling a bandwidth and spectral power of light emitted by the controlled light source;
b. measuring a light spectrum emitted by the controlled light source using the AHS sensor;
c. measuring, by using the AHS sensor, a light spectrum of reflected/backscattered light which was transmitted from the controlled light source and subsequently reflected/backscattered off the ore; and
d. comparing, by using a processor, the measured light spectrum emitted by the controlled light source with the measured light spectrum of the reflected/backscattered light.

It should be noted that the method does not require a “calibration” feature, such as a white card within a frame of view.

The AHS sensor may be directed towards a muck/veneer/stockpile/other area still to be excavated, in order to scan ore forming part of, or being located on, the muck/veneer/stockpile/other area still to be excavated.

The AHS sensor may be spaced about 5-30 m away from the ore being scanned. It should however be noted that by using a high power source or high sensitivity receiver, this range may by extended beyond 30 m, e.g. to 50 m/100 m (or even more).

The method may include utilising data/information obtained from the sensor in order to determine a value of the ore which has been scanned/sensed.

The method may include determining the value of the scanned/sensed ore by utilising the comparison of the measured light spectrum emitted by the controlled light source and the measured light spectrum of the reflected/backscattered light. The determining step may be performed using a processor.

The method may include utilising the determined ore value in order to determine:
where the scanned/sensed ore should be transported to; and/or
whether excavation operations should be focused at a particular area/location where the scanned/sensed ore is located.

The method may include communicating a decision as to (i) where the scanned/sensed ore should be transported to and/or (ii) whether excavation operations should be focused at the said particular area/location, to an operator of the excavator.

The communication step may include displaying information on a user interface which can be viewed by the operator.

In accordance with a second aspect of the invention there is provided a system for analysing ore during excavation, wherein the system includes:
at least one active hyperspectral sensing (AHS) sensor which is configured to scan/sense ore during excavation, wherein the AHS sensor is mounted/located at an excavation site where ore is being excavated and whereby the AHS sensor is directed towards a portion of the ore at the excavation site.

The system may include a light source which is configured to illuminate the ore. The light source may form part of the AHS sensor. The system may include a controller/processor which is configured to control the operation of the light source (hereinafter referred to as the “controlled light source”). The controller/processor may form part of the AHS sensor.

The controller/processor may be configured to:
a. control a power spectrum of the controlled light source; and
b. compare, using the AHS sensor, an emitted light spectrum of light emitted from the controlled light source with a light spectrum of reflected/backscattered light which is reflected/backscattered off the ore.

The controller/processor may be configured to compare spectral features and/or brightness of the emitted light spectrum with spectral features and/or brightness of the reflected/backscattered light.

The controller/processor:
a. control an operation of the controlled light source, which includes controlling a bandwidth and spectral power of light emitted by the controlled light source;
b. measure a light spectrum emitted by the controlled light source using the AHS sensor;
c. measure, by using the AHS sensor, a light spectrum of reflected/backscattered light which was transmitted from the controlled light source and subsequently reflected/backscattered off the ore; and
d. compare the measured light spectrum emitted by the controlled light source with the measured light spectrum of the reflected/backscattered light.

It should be noted that the method does not require a “calibration” feature, such as a white card within a frame of view.

The system may include a processing module which is configured to utilise data/information obtained from the sensor in order to determine a value of the ore which has been scanned/sensed by the sensor.

The processing module may be configured to utilise the determined ore value in order to determine:
where the scanned/sensed ore should be transported to; and/or
whether excavation operations should be focused at a particular area/location where the scanned/sensed ore is located.

A “module”, in the context of the specification, includes an identifiable portion of code, computational or executable instructions, or a computational object to achieve a particular function, operation, processing, or procedure. A module may be implemented in software, hardware or a combination of software and hardware. Furthermore, modules need not necessarily be consolidated into one device.

The controlled light source may be a supercontinuum beam. The controlled light source may be temporally variant to improve signal fidelity. The emitted light or light signal may be encoded to enable the use of correlation methods when comparing an emitted signal or emitted light with a received signal or a received light.

### BRIEF DESCRIPTION OF THE DRAWINGS

The invention will now be described, by way of example, with reference to the accompanying drawings. In the drawings:

FIG. 1 shows a schematic illustration of the system in accordance with the invention; and

FIG. 2 shows a schematic layout of the system shows in FIG. 1.

### DESCRIPTION OF PREFERRED EMBODIMENTS

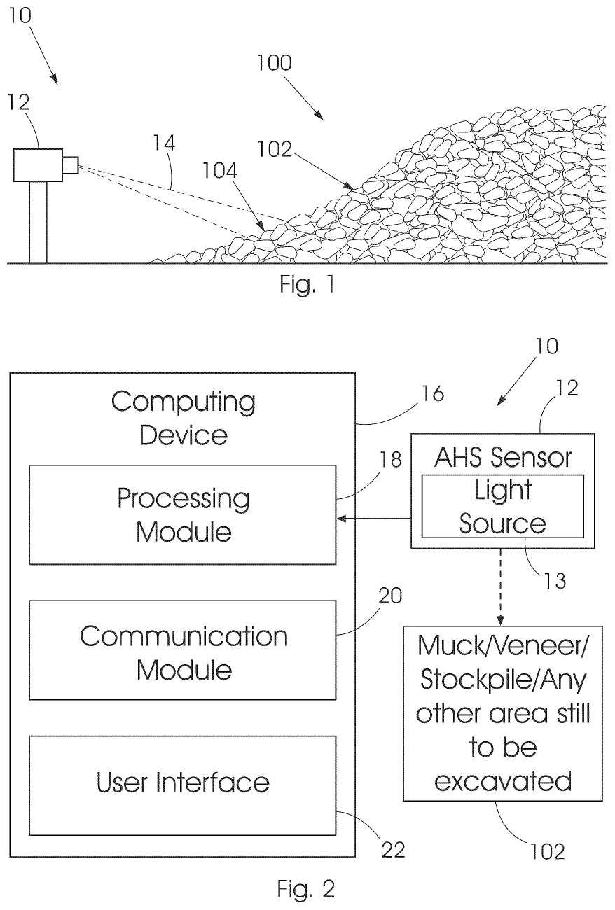

The present invention aims to address ore sensing during excavation, by providing a system 10 which uses standoff optical technology in the form of an active hyperspectral sensing (AHS) sensor 12. The AHS sensor 12 can be used/mounted/installed at an excavation site 100 (e.g. where excavators would typically be used to collect ore 104 and have it distributed to further downstream processing stations) and be used to scan portions of the ore at the excavation site 100 (e.g. ore 104 contained on a muck/veneer/stockpile/area still to be excavated 102. The AHS sensor 12 can be configured such that it can be moved and placed at different positions (e.g. to scan different parts of a veneer 102) (e.g. see FIG. 1).

Alternatively, the AHS sensor 12 could be installed on a vehicle/machine which can then move the sensor 12 to different areas at the excavation site 100 in order to scan different portions of the ore 104. In another alternative example, the AHS sensor 12 could be mounted on a raised stanchion or observation pole or structure, and look down into an operating pit. The AHS sensor 12 would typically use a whisk-broom approach in order to perform the scanning. However, it should be noted that it may also be possible, in future, to use a push-broom approach or possibly even snapshot implementations.

AHS sensors 12 use a controlled hyperspectral beam to generate a spotlight which can be carefully monitored to identify certain types of minerals (based on reflected or scattered light which is detected). More specifically, the system 10 includes a light source 13 which is configured to emit light towards ore 100 to be scanned. The light source 13 may be incorporated into the AHS sensor 12 or may be separate therefrom. The light source 13 (hereinafter referred to as the “controlled light source 13”) can typically be controlled by a controller/processor. The controller/processor is typically incorporated into the AHS sensor 12. However, in an alternative embodiment the controller/processor may be operatively connected to the AHS sensor 12.

The AHS sensor 12, together with its controller/processor, is configured to:
a. control an operation of the controlled light source 13, which includes controlling a bandwidth and spectral power of light emitted by the controlled light source 13;
b. measure a light spectrum emitted by the controlled light source 13 using the AHS sensor 12;
c. measure, by using the AHS sensor 12, a light spectrum of reflected/backscattered light which was transmitted from the controlled light source 13 and subsequently reflected/backscattered off the ore 104; and
d. compare the measured light spectrum emitted by the controlled light source 13 with the measured light spectrum of the reflected/backscattered light.

The controller/processor of the AHS sensor may be configured to compare spectral features and/or brightness of the emitted light spectrum with spectral features and/or brightness of the reflected/backscattered light.

The controlled light source 13 may be a supercontinuum beam. The controlled light source 13 may be temporally variant to improve signal fidelity. The emitted light or light signal may be encoded to enable the use of correlation methods when comparing an emitted signal or emitted light with a received signal or a received light.

The operation of AHS sensing is a known technology which has been described by others in the field. Reference is in this regard made to the following publication which is incorporated herein by reference:
Kääriäinen, Teemu, et. al., Active Hyperspectral Sensor Based on MEMS Fabry-Péron Interferometer, 2019 (Published 12 May 2019), Sensors, MDPI
https://www. researchgate. net/publication/333046518_Active_Hyper spectral_Sensor_Based_on_MEMS_Fabry-Perot_Interferometer

As mentioned, AHS employs a whisk-broom approach (although it is likely that AHS may, in future, use a push-broom approach), meaning that the AHS sensor 12 produces a beam (see reference numeral 14 which illustrates this beam schematically) which must be scanned over the surface of interest (e.g. the surface of the ore 100 exposed on the muck/veneer/stockpile/other area still to be excavated 102) to produce the desired output. The desired output can then be used (1) for fleet management and (2) feed-forward control, both of which are explained in more detail further below.

FIG. 1 shows a specific example where the AHS sensor 12 is mounted at a position near a muck/veneer/stockpile/other area still to be excavated 102 (e.g. about 5-30 m from the area to be scanned). By directing the AHS sensor 12 towards the area still to be excavated 102, portions of the ore 104 can be scanned and analysed.

By using an AHS sensor 12, it is possible to measure not only grade but also mineralogy, which leads to the opportunity to measure a value of the ore (i.e. ore value), which is a more sophisticated control quantity than grade alone. In this regard, it should be noted that ore value is a function not only of grade, but also of mineralogy and physical characteristics. For example, two rocks may be of the same grade, but one of them may have a higher hardness and will therefore require more energy intensive processing. As a result, the “harder” rock will be lower in value, when compared to the other rock.

### EXAMPLE

As will be clear from the explanation above, the system 10 allows for sensing during excavation at an excavation site 100. The AHS sensor 12 is used to scan a top portion of ore 104 (e.g. located on a muck/veneer/stockpile/other area still to be excavated 102) which is exposed to the sensor 12. The value of the scanned ore 104 can then be determined subsequently, by making use of the measurements taken by the AHS sensor 12. For example, the system 10 may include a computing device/system 16 which includes a processing module 18 and a communication module 20. The data/information obtained by the AHS sensor 12 is sent to the communication module 20 via a communication network/link (e.g. via wireless communication, such as Bluetooth). The processing module 18 then utilises the data/information to calculate the ore value. The computing device 16 may be located on an excavator which is used at the excavation site 100 or could be located at a remote location, e.g. a monitoring station. Based on the measured grade, mineralogy and/or ore value, a decision can be made as to whether:
(i) the scanned ore 104 may be of high value and further excavation is required at that particular location on the muck/veneer/stockpile/other area still to be excavated 102, in order to excavate and retrieve the high value ore 104 for further processing; or
(ii) the scanned ore 104 is of lower value and further excavation should rather focus on other areas on the muck/veneer/stockpile/other area still to be excavated 102.

In another example, the scanned ore which is of lower value could be transported to a waste pile or stockpile, while higher value ore could be transported to a crusher or leach pad for further processing.

The indication of the ore value can typically be communicated to an operator via a user interface 22. In one example, the user interface 22 may form part of the computing device 16 and may be installed inside a cab of an excavator. In a slight alternative example, the computing device 16 could send the indication of the ore value to a separate user interface 22 (e.g. via wireless communication) installed inside a cab of an excavator (e.g. the user interface 22 may include a display screen on which information is displayed). The user interface 22 would then indicate to the operator of an excavator (e.g. on a display screen) whether the area/location at which the ore 104 was scanned, should be prioritized for excavation (i.e. higher value ore), so that the ore 104 can be sent for further processing, or whether the operator should rather focus on excavating other areas.

It should be noted that the system of the present invention can be used to deliver fleet management instructions, provide a priori information for conventional belt-based bulk ore sorters, calibrate the mine control ore body model, and anticipate changes in the plant via feed-forward control.

It should also be noted that with bulk sorters, not all BOS (bulk ore sorting) sensing is deterministic. A given BOS sensor might measure one aspect of value (such as clay content) and rely on the input of LIBS data for grade. Alternatively, clay and geographic data obtained during excavation might feed-forward to inform a BOS system which proxy to use if direct grade measurement is not possible. Proxy guidance could be critical in PGM's (Platinum Group Metals), where grade is measured in PPM (parts per million).

Plant stabilization mitigation action can be taken through a variety of approaches including using talc data from the AHS sensor. Talc tends to cause overflow in the flotation plant. If talc can be detected ahead of processing, mitigation action can be taken by changing the reagent concentration in the plant.

1. A method of analysing ore during excavation, wherein the method includes:
during excavation, scanning/sensing ore by using an active hyperspectral sensing (AHS) sensor.

2. The method of claim 1, wherein the scanning/sensing step includes:
a. controlling a power spectrum of a light source (hereinafter referred to as the “controlled light source”); and
b. comparing, by using the AHS sensor, an emitted light spectrum of light emitted from the controlled light source with a light spectrum of reflected/backscattered light which is reflected/backscattered off the ore.

3. The method of claim 1, wherein the scanning/sensing step includes:
a. controlling an operation of the controlled light source, which includes controlling a bandwidth and spectral power of light emitted by the controlled light source;
b. measuring a light spectrum emitted by the controlled light source using the AHS sensor;
c. measuring, by using the AHS sensor, a light spectrum of reflected/backscattered light which was transmitted from the controlled light source and subsequently reflected/backscattered off the ore; and
d. comparing, by using a processor, the measured light spectrum emitted by the controlled light source with the measured light spectrum of the reflected/backscattered light.

4. The method of claim 3, wherein the AHS sensor is directed towards a muck/veneer/stockpile in order to scan ore forming part of, or being located on, the muck/veneer/stockpile.

5. The method of claim 3, wherein the AHS sensor is spaced about 5-30 m away from the ore being scanned.

6. The method of claim 3, which includes determining a value of the scanned/sensed ore by utilising the comparison of the measured light emitted and the reflected/scattered light.

7. The method of claim 6, which includes utilising the determined ore value in order to determine:
where the scanned/sensed ore should be transported to; and/or
whether excavation operations should be focused at a particular area/location where the scanned/sensed ore is located.

8. The method of claim 7, which includes communicating a decision as to (i) where the scanned/sensed ore should be transported to and/or (ii) whether excavation operations should be focused at the said particular area/location, to an operator of the excavator.

9. The method of claim 8, wherein the communication step includes displaying information on a user interface which can be viewed by the operator.

10. A system for analysing ore during excavation, wherein the system includes:
at least one active hyperspectral sensing (AHS) sensor which is configured to scan/sense ore during excavation, wherein the AHS sensor is mounted/located at an excavation site where ore is being excavated and whereby the AHS sensor is directed towards a portion of the ore at the excavation site.

11. The system of claim 10, which includes a light source which forms part of the AHS sensor and a controller/processor which forms part of the AHS sensor, wherein the controller/processor is configured to control the operation of the light source (hereinafter referred to as the “controlled light source”).

12. The system of claim 11, wherein the controller/processor is configured to:
a. control a power spectrum of the controlled light source; and
b. compare, using the AHS sensor, an emitted light spectrum of light emitted from the controlled light source with a light spectrum of reflected/backscattered light which is reflected/backscattered off the ore.

13. The system of claim 11, wherein the controller/processor is configured to:
a. control an operation of the controlled light source, which includes controlling a bandwidth and spectral power of light emitted by the controlled light source;
b. measure a light spectrum emitted by the controlled light source using the AHS sensor;
c. measure, by using the AHS sensor, a light spectrum of reflected/backscattered light which was transmitted from the controlled light source and subsequently reflected/backscattered off the ore; and
d. compare the measured light spectrum emitted by the controlled light source with the measured light spectrum of the reflected/backscattered light.

14. The system of claim 13, which includes a processing module which is configured to utilise data/information obtained from the sensor in order to determine a value of the ore which has been scanned/sensed by the sensor.

15. The system of claim 14, wherein the processing module is configured to utilise the determined ore value in order to determine:
where the scanned/sensed ore should be transported to; and/or
whether excavation operations should be focused at a particular area/location where the scanned/sensed ore is located.
